(19) United States
(12) Plant Patent Application Publication
(10) Pub. No.: US 2006/0095999 P1
(43) Pub. Date: May 4, 2006

(54) ASTER PLANT NAMED 'CEFORTUNA'

(50) ASTER NOVI-BELGII; ASTER

(75) Inventors: Harry W.M. van Straalen, Rijsenhout (NL); Wilfried J. Poland, Wervershoof (NL)

Correspondence Address: CHARLES A. WENDEL;STEPTOE & JOHNSON LLP, 1330 CONNECTICUT AVENUE NW, WASHINGTON, DC, 20036, US

(73) Assignee: Chrysanthemum Breeders Association N.V., Aalsmeer (NL)

(21) Appl. No.: 10/979,190

(22) Filed: Nov. 3, 2004

(51) Int. Cl. A01H 5/00

(52) U.S. Cl. PLT/355

(57) ABSTRACT

An Aster plant named ‘Cefortuna’ characterized by its medium sized blooms with purple ray-florets and yellow disc florets, which can be propagated by means of cuttings from cuttings and produced with a short period.

### RELATED CULTIVARS

‘Cefortuna’ is developed from a breeding program for pot asters which has so far yielded the commercial varieties ‘Dynastaster’ (U.S. Plant patent apending), ‘Diamaster’ (U.S. Plant patent pending) and Dukaster (U.S. Plant patent pending).

### BACKGROUND OF THE INVENTION

‘Cefortuna’ is a product of a breeding-program which had the objective of creating new Aster cultivars, which can be grown as pot plants and propagated by means of cuttings from cuttings, similar to the cultivation and propagation of all year round chrysanthemum. The new plant of the present invention comprises a new and distinct cultivar of an Aster plant. ‘Cefortuna’ is a seedling from a cross of a breeding program maintained at Chrysanthemum Breeders Association Research BV, Rijsenhout, Holland. The female parent is 97.6128, a non-commercialized aster variety; the male parent is unknown, being a mixed population of a group of male parents. A comparison with parent aster 97.6128 is also given in this application. The new and distinct cultivar was discovered and selected as a flowering plant within the progeny of the stated cross by Harry W. M. van Straalen. in a controlled environment (greenhouse) in Rijsenhout, Holland in 2000. The first act of asexual reproduction of ‘Cefortuna’ was accomplished when vegetative cuttings were taken from the initial selection in 2000 in a controlled environment in Rijsenhout, Holland.

### SUMMARY OF THE INVENTION

The present invention is a new and distinct variety of Aster bearing medium sized blooms with purple ray-florets and yellow disc florets, which can be propagated by a cutting from a cutting and produced as pot plants in 8 weeks time.

### BRIEF DESCRIPTION OF THE DRAWINGS

The present invention of a new and distinct variety of Aster is shown in the accompanying drawings, the color being as nearly true as possible with color photographs of this type.

FIG. 1 shows a plant of the cultivar in full bloom.

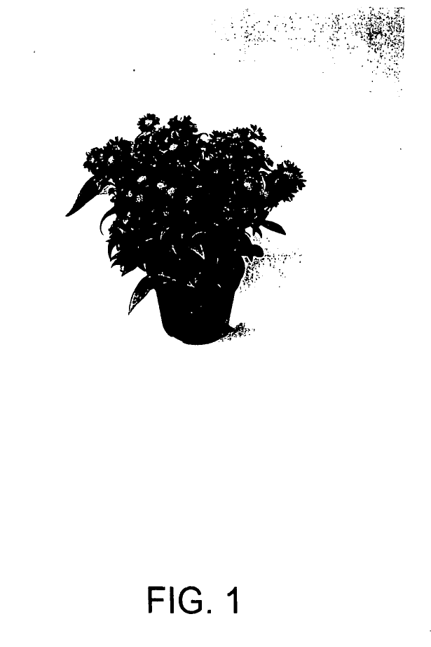

FIG. 2 shows the various stages of bloom of the new cultivar.

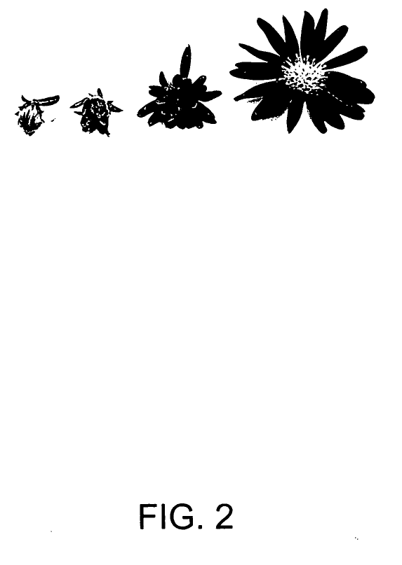

FIG. 3 shows the foliage of the new cultivar.

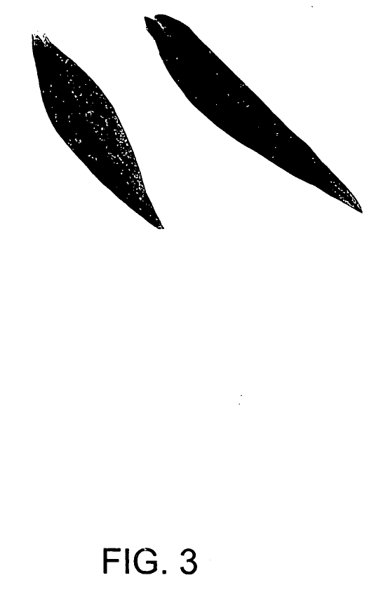

### DESCRIPTION OF THE INVENTION

This new variety of Aster is of the botanical classification Aster novi-belgii L. The observations and measurements were gathered from plants grown in a greenhouse in Rijsenhout Holland in a photo-periodic controlled crop under conditions generally used in commercial practice. The greenhouse temperatures during this crop were at day-time between 18° C. and 25° C. and at night 20° C. The photo-periodic response time in this crop was 39 days, after an average of 16 long days after sticking of the unrooted cuttings. Plants are pinched 10 days after sticking. Growth retardants were applied in an average dose of 1.5 gram/liter water, starting one week after pinching. The plants were observed (directly) during the flowering of this crop. The plant is susceptible to Powdery Mildew. No tests were done on cold or drought tolerance. This new variety produces medium sized blooms with purple ray-florets and yellow disc-florets blooming on the plant for 4 weeks. This new variety of Aster has been found to retain its distinctive characteristics throughout successive propagations, however the phenotype may vary significantly with variations in environment such as light intensity and temperature. ‘Cefortuna’ can be planted with assimilation lightning (high pressure sodium lamps) between week 5 and week 35 under greenhouse conditions in Holland.

From the cultivars known to inventor the most similar existing cultivar in comparison to ‘Cefortuna’ are its female parent 97.6128 and the varieties ‘Dukaster’ and ‘Dynaster’. When these varieties are being compared with ‘Cefortuna’ the following differences are noticed: The differences between ‘Cefortuna’, 97.6128, ‘Dukaster’ and ‘Dynaster’ are (1) Flower color. (2) Growth habit. (3) Leaf width. (1) The color of the ray-florets of ‘Cefortuna’ is purple, while it is pink in ‘Dukaster’ and violet in 97.6128 and ‘Dynaster’. (2) The growth habit is upright and partially spreading in ‘Cefortuna’, while this is upright in 97.6128, ‘Dukaster’ and ‘Dynaster’ (3) The leaves of ‘Cefortuna’ and ‘Dynaster’ are of intermediate width, while those of 97.6128 are smaller, and those of ‘Dukaster’ are broader.

The following is a description of the plant and characteristics that distinguish ‘Cefortuna’ as a new and distinct variety. The color designations are taken from the plant itself. Accordingly, any discrepancies between the color designations and the colors depicted in the photographs are due to photographic tolerances. The color chart used in this description is: The Royal Horticultural Society Colour Chart, edition 1995.

TABLE 1
Botanical Description of cultivar ‘Cefortuna’

| | |
|---|---|
| Bud | |
| Size | Medium; Cross-section 0.3 cm, height |
| | 0.5 cm |
| Outside color | Purple-violet 81C |
| Involucral bracts | 2 rows, length 4 mm, width 3 mm |
| Involucral bracts | Not present |
| among disc-florets | |
| Involucral bracts color | Green 138 D |
| Bloom | |
| Type | Daisy |
| Size | Medium |
| Fully expanded | 2.5-3 cm |
| Number of blooms per branch | 10-12 |
| Peduncle length | 3 cm |
| Peduncle color | Green 139 B |
| Peduncle angle | 45° |
| Performance on the plant | 4 weeks |
| Seeds | Produced in small quantities, |
| | oval/ovate shaped, grey-brown |
| | 199 A, 1 mm. in length |
| Color | |
| Center of the bloom | Immature Yellow-green 150C |
| (disc-florets) | Mature Yellow 1A |
| Color of the ray-florets | Upper surface: Red-purple 72B |
| | Lower surface: Purple-violet 81C |
| Tonality from Distance | A pot aster with purple flowers and |
| | a yellow disc |
| Color of the upper | Red-purple 72C |
| surface of the ray-florets | |
| after aging of the plant | |
| Ray florets | |
| Number of whorls of | 2 |
| ray-florets | |
| Texture | Upper and under side smooth |
| Number of ray-florets | 20-30 |
| Shape in cross-section | Straight |
| Curvature of longitudinal axis | Flat |
| Length of corolla tube | 0.3 cm |
| Ray-floret length | 1.5 cm |
| Ray-floret width | 0.2 cm |
| Shape of tip | Pointed |
| Shape of apex | Acute |
| Disc florets | |
| Disc diameter | 0.7 cm |
| Distribution of disc florets | Numerous, clearly visible at all stages of |
| | flowering |
| Shape | Tubular |
| Lenght | 0.6 cm |
| Color | Yellow-green 145 D |
| Reproductive Organs | |
| Stamen | Present in Disc florets only |
| Stamen color | Yellow 13 A |
| Pollen color | Yellow-orange 14 A |
| Styles | Present in both Ray- and Disc-florets |
| Style color | Yellow-green 150 D |
| Style length | 0.6 cm |
| Ovaries | Enclosed in calyx |
| Calyx shape | Pappus |
| Calyx lenght | 0.4 cm |
| Calyx color | Yellow-green 150 D |
| Plant | |
| Form | A pot aster meant for indoor use |
| Growth habit | Upright and partially spreading |
| Growth rate | Vigorous |
| Height | 22 cm |
| Width | 17 cm |
| Internode lenght | 1.5-2 cm |
| Stem diameter | 2-3 mm |
| Stem color | Green 139 B |
| Stem strength | Strong |
| Stem brittleness | Not brittle |
| Stem anthocyanin coloration | Absent |
| Length of lateral branch | From top to bottom 18 cm |
| Lateral branch color | Green 139 B |
| Lateral branch, attachment | 30-40° |
| Branching (average number of | Medium with 3 breaks after pinching |
| lateral branches) | |
| Internode length | 1 cm |
| Flowering Response (photo- | 39 Days |
| periodic controlled crop, | |
| not natural season) | |
| Foliage | |
| Leaf arrangement | Alternate |
| Color immature stage | Upper side Yellow-green 144 C |
| | Under side Yellow-green 146 D |
| Color mature stage | Upper side Green 137 B |
| | Under side Green 139 C |
| Color Midvein | Upper side Yellow-green 147 D |
| | Under side Yellow-green 147 C |
| Size | Medium; length 5-7 cm, width 1-1.5 cm |
| Quantity (number per lateral | 12 |
| branch) | |
| Shape | Elliptic |
| Texture upper side | Glabrous |
| Texture under side | Glabrous |
| Venation arrangement | Pinnately netted |
| Shape of the margin | Sinuate |
| Shape of Base | Attenuate |
| Apex | Acuminate |

TABLE 2
Differences with the comparison varieties

| | ‘Cefortuna’ | 97.6128 | ‘Dukaster’ | ‘Dynaster’ |
|---|---|---|---|---|
| Color upper | Red-purple | Violet-blue | Purple-violet | Violet-blue |
| side ray-florets | 72B | 92A | 81A | 90A |
| Growth habit | Upright and | Upright | Upright | Upright |
| | spreading | growing | growing | growing |
| Leaf width | 1-1.5 cm | 0.6-1.2 cm | 2 cm | 1.5 cm |

I claim:

1. A new and distinct variety of Aster plant as described and illustrated.
